(19) United States
(12) Patent Application Publication
(10) Pub. No.: US 2007/0134784 A1
(43) Pub. Date: Jun. 14, 2007

(54) MICROREPLICATED MICROARRAYS

(76) Inventors: Kurt J. Halverson, Lake Elmo, MN (US); Mark F. Schulz, Lake Elmo, MN (US); Jerald K. Rasmussen, Stillwater, MN (US); Paul D. Graham, Woodbury, MN (US)

Correspondence Address: 3M INNOVATIVE PROPERTIES COMPANY, PO BOX 33427, ST. PAUL, MN, 55133-3427, US

(21) Appl. No.: 11/275,099

(22) Filed: Dec. 9, 2005

(51) Int. Cl. C12M 1/34

(52) U.S. Cl. 435/287.2

(57) ABSTRACT

A microarray comprises a microstructured surface and an attachment chemistry layer disposed on at least a portion of the microstructured surface, the microstructured surface comprising primary microstructured elements comprising walls.

### FIELD

This invention relates to microarrays that are useful, for example, in applications such as gene sequencing and combinatorial chemistry and, in another aspect, to methods of detecting analytes using the microarrays.

### BACKGROUND

Microarrays can be used in a variety of applications such as, for example, gene sequencing, monitoring gene expression, gene mapping, bacterial identification, drug discovery, biomarker identification, and combinatorial chemistry.

Microarrays are typically produced on planar substrates (for example, silicon wafers or glass microscope slides). Microarray features are typically printed or “spotted” onto the substrate using liquid deposition techniques. The microarray features are separated by “unprinted” space between each feature. The amount of space between each feature, however, is dependent upon the technique used to manufacture the microarray.

In general, microarrays produced using spotting techniques have a relatively low feature density. Manufacturing techniques involving spotting of micro-volume droplets on the array surface, for example, generally require sufficient space in between features so that adjacent droplets do not contact each other during manufacturing (see, for example, U.S. Pat. No. 6,613,893 (Webb)). Control of spot size, shape, and drying pattern are dependent upon factors such as the volume deposited, the viscosity of the solution, the wetting behavior of the solution on the surface, and environmental conditions (for example, temperature and humidity) during manufacturing. Efforts to confine liquid to predefined areas have included, for example, the use of alternating hydrophilic and hydrophobic patterns (see, for example, U.S. Pat. No. 5,474,796 (Brennan) and U.S. Pat. No. 6,630,358 (Wagner et al.)), placing the liquid on the top surface of three dimensional posts (see, for example, U.S. Pat. No. 6,454,924 (Jedrzejewski et al.)), and containing the liquid with porous regions in a solid non-porous matrix (see, for example, U.S. Pat. No. 6,383,748 (Carpay et al.)).

### SUMMARY

In view of the foregoing, we recognize that there is a need for high density microarrays produced using spotting techniques.

Briefly, the present invention provides high density microarrays that can be produced using spotting techniques such as, for example, inkjet, piezo, or contact printing. The microarrays comprise a microstructured surface and an attachment chemistry layer disposed on at least a portion of the microstructured surface. The microstructured surface comprises primary microstructured elements comprising walls.

In the microarrays of the invention, the wall thickness represents the unprinted space between features. The microarrays of the invention therefore have very little space between features. In addition, the microarrays of the invention can include feature shapes that are typically unattainable via directing printing on a planar substrate (for example, densely packed squares).

In the microarrays of the invention, the attachment chemistry layer allows for the subsequent affixation of reactants thereto. Where affixation via covalent bonding is desired, larger volumes of reactant can be placed in the “microcompartments” formed by the walls of the microarray without increasing the size (that is, projected area or “footprint”) of the feature. The amount of reactant available to bond to the surface for a given area is therefore increased.

Another advantage of the microarrays of the invention is the enablement of higher sensitivity detection via the use of enzyme-linked detection protocols. By confining the solution within a microcompartment, the product of the resulting enzymatic reaction (for example, a low molecular weight fluorescent molecule) is prevented from diffusing beyond the microcompartment walls.

In addition, the uniform features (defined by the walls) of the microarrays of the invention simplify the processing of the microarray image. Minimal user intervention is necessary during image processing.

Typically, microarrays produced using spotting techniques require very precise registration when the microarray features are spotted onto the substrate. The microarrays of the invention allow more tolerance during spotting. Provided that a droplet is deposited within a microcompartment, it will distribute within the microcompartment, but still be confined within the microcompartment walls. It does not matter exactly where within the microcompartment the droplet lands.

Thus, the microarrays of the invention meet the need for high density microarrays produced using spotting techniques.

In another aspect, this invention provides a kit comprising a microarray of the invention and a cover.

In yet another aspect, this invention also provides a method for detecting analytes in a sample. The method involves (a) providing a microarray of the invention wherein a reactant is affixed to the attachment chemistry layer, (b) depositing a sample into at least one primary microstructured element of the microarray such that the sample contacts the reactant and forms a complex, (c) contacting the complex with a second reactant to form a ternary complex, (d) detecting any ternary complexes, and (e) relating the presence or amount of the ternary complexes to the presence or amount of analyte in the sample.

### DEFINITIONS

For purposes of this invention, the following definitions shall have the meanings set forth.

“Affix” shall, with respect to reactants and an attachment chemistry layer, include any mode of attaching reactants to an attachment chemistry layer. Such modes shall include, without limitation, covalent and ionic bonding, adherence such as with an adhesive, and physical entrapment within an attachment chemistry layer. In the case of linking agents, reactants may be affixed to the attachment chemistry layer by linking agents that are created by functionalizing a surface, such as with an acid wash, or by linking agents that are coated.

“Amphoteric” shall mean, with respect to any molecule, compound, composition, or complex, having character of both an acid and a base. The term includes molecules, compounds, compositions, or complexes that are both anionic and cationic (for example, a polypeptide at its isoelectric point).

“Analyte” shall mean a molecule, compound, composition, or complex, either naturally occurring or synthesized, to be detected or measured in or separated from a sample of interest. Analytes include, without limitation, proteins, peptides, amino acids, fatty acids, nucleic acids, carbohydrates, hormones, steroids, lipids, vitamins, bacteria, viruses, pharmaceuticals, and metabolites.

“Array” shall mean a tool that is useful for any chemical or biochemical analysis and that consists of isolated regions or “sample spots” provided in an orderly arrangement. Arrays can be useful, for example, in gene sequencing, monitoring gene expression, bacterial identification, drug discovery, biomarker identification, combinatorial chemistry, and the like. “Microarrays” typically have isolated regions or sample spots that are less than about 1 square millimeter in area, sometimes less than about 0.25 square millimeters in area or even less than about 0.04 square millimeters in area.

“Attachment chemistry layer” shall mean any layer, surface, or coating that can immobilize, affix, or reversibly affix reactants thereto. An attachment chemistry layer can, for example, be a coating disposed on a microstructured surface or it can be a functionalized portion of a microstructured surface.

“Bifunctional” shall mean, with respect to any molecule, compound, composition or complex, having more than one functional group. For example, a bifunctional molecule can have an amino group capable of forming a covalent bond with an azlactone moiety and an anionic group capable of forming an ionic bond with a cation.

“Binding site” shall mean a discrete location disposed on an attachment chemistry layer wherein reactants can affix thereto.

“Complementary functional group” shall mean a group capable of reacting with a recited group to form an ionic bond, covalent bond, or combinations thereof. For example, the complementary functional group can be a group on an attachment chemistry layer capable of reacting with group X¹ in Formulas I, II, or III.

“Functional group” shall mean a combination of atoms in a molecule, compound, composition, or complex that tends to function as a single chemical entity. Examples of functional groups include, but are not limited to, —NH₂ (amine), —COOH (carboxyl), siloxane, —OH (hydroxyl), and azlactone.

“Ionic” shall mean any chemical species that has a formal charge, that is, has an excess (negative formal charge) or a deficiency (positive formal charge) of electrons on at least one atom of the species. A polymeric surface is “ionic” if it contains at least one chemical species having a formal charge even if the polymeric coating is associated with a counterion (for example, in solution) having an opposite formal charge. The counterion may produce a surface with a net neutral charge even though the polymer surface itself has a formal positive or negative charge.

“Linking agent” shall mean any chemical species capable of affixing a reactant to the attachment chemistry layer.

“Microstructured elements” shall mean a recognizable geometric shape that either protrudes or is depressed.

“Primary microstructured elements” shall mean a microstructured element on a surface, the primary microstructured element having the largest scale of any microstructured element on the same surface.

“Secondary microstructured elements” shall mean a smaller scale microstructured element on the same surface as the primary microstructured element.

“Reactant” shall mean any chemical molecule, compound, composition or complex, either naturally occurring or synthesized, that is capable of binding an analyte in a sample of interest either alone of in conjunction with a molecule or compound that assists in binding the analyte to the attachment chemistry layer, such as, for example, a coenzyme. The reactants of the present invention are useful for chemical or biochemical measurement, detection or separation. Examples of reactants include, without limitation, amino acids, nucleic acids, including oligonucleotides and cDNA, carbohydrates, and proteins such as enzymes and antibodies.

“Tethering compound” shall mean a compound that has two reactive groups. One of the groups (that is, the substrate-reactive functional group) can react with a complementary functional group on the surface of a substrate (for example, the microstructured surface or any intervening layer between the attachment chemistry layer and the microstructured surface) to form a tethering group. The other reactive group (that is, the N-sulfonylaminocarbonyl group or the N-sulfonyldicarboximide group) can react with an amine-containing material. Reaction of both reactive groups of the tethering compound results in the formation of a connector group between the substrate and an amine-containing material (that is, the amine-containing material can be immobilized on the substrate).

“Tethering group” shall mean a group attached to a substrate that results from the reaction of a compound that has two reactive groups with a complementary functional group on the surface of the substrate with a tethering compound. The tethering group includes a N-sulfonylaminocarbonyl group or a N-sulfonyldicarboximide group.

### BRIEF DEESCRIPTION OF THE DRAWINGS

FIG. 1 is a cross-sectional view of a microarray of the invention.

FIG. 2 is a cross-sectional view of another microarray of the invention.

FIG. 3 is a scanning electron micrograph photograph of a film having a microstructured surface described in Preparative Example 1.

FIG. 4 is an image of a fluorescent pattern in a film having a microstructured surface described in Preparative Example 1.

### DETAILED DESCRIPTION

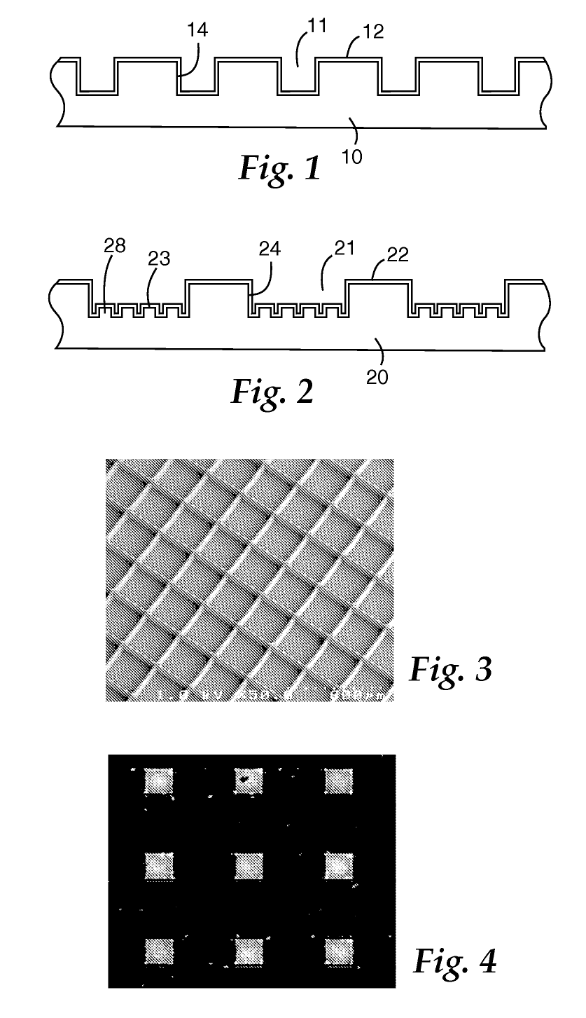

FIG. 1 illustrates an example of a microarray of the present invention. The microarray comprises a microstructured surface 10 and an attachment chemistry layer 12. The microstructured surface 10 comprises primary microstructured elements 11 (depicted in FIG. 1 as depressed microstructured elements) comprising walls 14. Although the attachment chemistry layer is depicted in FIG. 1 as covering the entire microstructured surface, in practice the attachment chemistry layer may be disposed primarily on only a portion of the microstructured surface.

Microstructured Surface

The microstructured surface comprises primary microstructured elements comprising walls. The walls generally have a thickness of between about 1 and about 50 micrometers; preferably between about 1 and about 30 micrometers; more preferably between about 5 and about 30 micrometers.

In general, the geometrical configuration of the primary microstructured elements is chosen to have sufficient capacity to control placement of an individual drop or a certain volume of solution containing the reactant or analyte. In some embodiments, the geometrical configuration is chosen such that the microstructured element pitch (that is, center to center distance between microstructured elements) is between about 1 and about 1,000 micrometers; preferably between about 10 and about 500 micrometers; more preferable between about 50 and about 400 micrometers.

The primary microstructured elements can have any structure. For example, the structure for the primary microstructured elements can range from the extreme of cubic elements with parallel vertical, planar walls, to the extreme of hemispherical elements, with any possible solid geometrical configuration of walls in between the two extremes. Specific examples include cube elements, cylindrical elements, conical elements with angular, planar walls, truncated pyramid elements with angular, planar walls, honeycomb elements and cube corner shaped elements. Other useful microstructured elements are described in PCT Publications WO 00/73082 and WO 00/73083.

The pattern of the topography can be regular, random, or a combination of the two. “Regular” means that the pattern is planned and reproducible. “Random” means one or more features of the microstructured elements are varied in a non-regular manner. Examples of features that are varied include, for example, microstructured element pitch, peak-to-valley distance, depth, height, wall angle, edge radius, and the like. Combination patterns can, for example, comprise patterns that are random over an area having a minimum radius of ten microstructured element widths from any point, but these random patterns can be reproduced over larger distances within the overall pattern. The terms “regular”, “random”, and “combination” are used herein to describe the pattern imparted to a length of web by one repeat distance of the tool having a microstructured pattern thereon. For example, when the tool is a cylindrical roll, one repeat distance corresponds to one revolution of the roll. In another embodiment, the tool can be a plate and the repeat distance would correspond to one or both dimensions of the plate.

The volume (that is, the void volume defined by a microstructured element) of a primary microstructured element can range from about 1 to about 20,000 picoliters (pL); preferably from about 1 to about 10,000 pL. Certain embodiments have a volume from about 3 to about 10,000 pL; preferably from about 30 to about 10,000 pL; more preferably from about 300 to about 10,000 pL.

Another way to characterize the structure of the primary microstructured elements is to describe the microstructured elements in terms of aspect ratios. An “aspect ratio” is the ratio of the depth to the width of a depressed microstructured element or the ratio of height to width of a protruding microstructured element. Useful aspect ratios for a depressed microstructured element typically range from about 0.01 to about 2; preferably from about 0.05 to about 1; more preferably from about 0.05 to about 0.8. Useful aspect ratios for a protruding microstructured element typically range from about 0.01 to about 15; preferably from about 0.05 to about 10; more preferably from about 0.05 to about 8.

The overall height of the primary microstructured elements depends on the shape, aspect ratio, and desired volume of the microstructured elements. The height of a microstructured element can range from about 5 to about 200 micrometers. In some embodiments, the height ranges from about 20 to about 100 micrometers; preferably from about 30 to about 90 micrometers.

Primary microstructured element pitch is typically in the range of from about 1 to about 1,000 micrometers. Certain embodiments have a primary microstructured element pitch of from about 10 to about 500 micrometers; preferably from about 50 to about 400 micrometers. The microstructured element pitch can be uniform, but it is not always necessary or desirable for the pitch to be uniform. In some embodiments, it may not be necessary, or desirable, that uniform microstructured element pitch be observed, nor that all features be identical. Thus, an assortment of different types of features, for example, microstructured elements with an assortment of microstructured element pitches may comprise the microstructured surface of the microarrays of the invention. The average peak to valley distances of individual elements is generally from about 1 to about 200 micrometers.

As depicted in FIG. 2, in some embodiments, the microarrays of the invention comprise secondary microstructured elements 28 that can, for example, improve wetting/uniform liquid distribution within each microstructured element. The primary microstructured elements 21 have a base surface 23 extending between the walls 24. The primary microstructured element base 23 can, for example, comprise secondary microstructured elements 28 (preferably, the secondary microstructured elements extend from one wall to a second wall). Although the attachment chemistry layer 22 is depicted in FIG. 2 as covering the entire microstructured surface 20, in practice the attachment chemistry layer may be disposed primarily on only a portion of the primary or secondary microstructured elements. The secondary microstructured elements have dimensions in the x-direction (that is, generally perpendicular to the base surface), as well as length and width. Generally, the x-direction dimension is between about 0.1 and about 50 micrometers; preferably between about 0.1 and about 20 micrometers. In some embodiments, the x-direction dimension is between about 0.1 and about 10 micrometers; preferably between about 0.1 and about 5 micrometers.

In some embodiments, the secondary microstructured element x-direction dimension is at least about 5 micrometers less than the height of the primary microstructured walls. For example, the secondary microstructured element x-direction dimension is at least 20 micrometers less than the height of the primary microstructured walls. In specific embodiments, the secondary microstructured element x-direction dimension is at least 50 micrometers less than the height of the primary microstructured walls; preferably at least 70 micrometers less.

The secondary microstructured elements can form any pattern such as, for example, any combination of parallel elements, nonparallel elements, or parallel and nonparallel elements. The secondary microstructured elements can intersect at any number of points, for example, straight parallel elements, and elements that meet at 90 degree angles.

In some embodiments, the secondary microstructured elements additionally have a volume (for example, a volume defined by secondary microstructured elements that intersect at 90 degrees or a volume defined by the secondary microstructured elements and an intersection with the primary microstructured walls). In such embodiments, the ratio of the volume of the primary microstructured elements to the volume of one secondary microstructured element is between about 5 and about 2,000,000. For example, the ratio can be between about 50 and about 1,000,000; preferably between about 150 and about 150,000; more preferably between about 35 and about 500.

The microstructured surface typically comprises a polymer, however it can comprise glass or any other material that is amenable to the coating, casting, or compressing techniques described below. Preferably, the microstructured surface comprises a material that does not interfere with the electromagnetic signal emitted by the desired analyte in response to excitation energy (for example, a material that does not transmit excitation energy or electromagnetic energy that is similar to the electromagnetic signal emitted by the desired analyte in response to excitation energy). Examples of electromagnetic signals that can be emitted by analytes include fluorescence, absorbance, electric current, chemiluminescence, and the like.

Nonlimiting examples of polymeric films useful for the microstructured surface include thermoplastics such as polyolefins (for example, polypropylene or polyethylene), poly(vinyl chloride), copolymers of olefins (for example, copolymers of propylene), copolymers of ethylene with vinyl acetate or vinyl alcohol, fluorinated thermoplastics such as copolymers and terpolymers of hexafluoropropylene and surface modified versions thereof, poly(ethyl terephthalate) and copolymers thereof, polyurethanes, polyimides, acrylics, and filled versions of the above using fillers such as silicates, silica, aluminates, feldspar, talc, calcium carbonate, titanium dioxide, and the like. Also useful are coextruded films and laminated films made from the materials listed above. Preferably, the microstructured surface comprises polyvinyl chloride, polyethylene, polypropylene, or copolymers thereof.

The microstructured surface can be made in a number of ways, such as using casting, coating, or compressing techniques. For example, microstructuring of the microstructured surface can be achieved by at least any of (1) casting a molten thermoplastic using a tool having a microstructured pattern, (2) coating of a fluid onto a tool having a microstructured pattern, solidifying the fluid, and removing the resulting film, or (3) passing a thermoplastic film through a nip roll to compress against a tool having a microstructured pattern. The tool can be formed using any of a number of techniques known to those skilled in the art, selected depending in part upon the tool material and features of the desired topography. Illustrative techniques include etching (for example, via chemical etching, mechanical etching, or other ablative means such as laser ablation or reactive ion etching, etc.), photolithography, stereolithography, micromachining, knurling (for example, cutting knurling or acid enhanced knurling), scoring or cutting, etc. Alternative methods of forming the microstructured surface include thermoplastic extrusion, curable fluid coating methods, and embossing thermoplastic layers, which can also be cured.

The extrusion method involves passing an extruded material or preformed substrate through a nip created by a chilled roll and a casting roll engraved with an inverse pattern of the desired microstructure. Or, an input film is fed into an extrusion coater or extruder. A polymeric layer is hot-melt coated (extruded) onto the input film. The polymeric layer is then formed into a microstructured surface.

Calendering can be accomplished in a continuous process using a nip, as is known in the film handling arts. In the present invention, a web having a suitable surface, and having sufficient thickness to receive the desired microstructured pattern is passed through a nip formed by two cylindrical rolls, one of which has an inverse image to the desired structure engraved into its surface. The surface layer contacts the engraved roll at the nip to form the microstructured pattern.

Attachment Chemistry Layer

The microarrays of the invention include an attachment chemistry layer disposed on at least a portion of the microstructured surface. The attachment chemistry layer is suitable for the subsequent affixation of reactants thereto.

A wide variety of attachment chemistry layers can be useful in the microarrays of the invention, provided the attachment chemistry layer is suitable for affixing reactants and is compatible with the assays and attendant conditions that are to be conducted on the particular micro array.

In some embodiments, the attachment chemistry layer is functionalized such that it comprises linking agents. The linking agents can be selected based on the reactants to be affixed to the microarray and the application for which the microarray will be used. Preferred linking agents include azlactone moieties such as those provided by copolymers taught in U.S. Pat. No. 4,304,705 (Heilmann et al.), U.S. Pat. No. 4,451,619 (Heilmann et al.), U.S. Pat. No. 5,262,484 (Coleman et al.), U.S. Pat. No. 5,344,701 (Gagnon et al.), and U.S. Pat. No. 5,403,902 (Heilmann et al.), all of which are incorporated herein by reference. Especially preferred copolymers are those prepared using hydrophilic or water-soluble comonomers such as acrylamide and acrylamide derivatives, hydroxyethylacrylate and methacrylate, and the like.

In addition to the azlactone copolymers set forth above, suitable azlactone functional compounds include those such as are disclosed in U.S. Pat. No. 4,485,236 (Rasmussen et al.) and U.S. Pat. No. 5,149,806 (Moren et al.), the disclosures of which are incorporated herein by reference.

Azlactone-functional hydrogel coatings such as those disclosed in U.S. Pat. No. 6,794,458 (Haddad et al.), which is incorporated herein by reference, can also be utilized. Azlactone-functional hydrogels can be prepared by first preparing a solution of a hydrophilic, azlactone-functional copolymer (for example, one of the azlactone-functional copolymers described above). This copolymer is then formulated with an appropriate crosslinker, and the mixture is then coated or applied to the microstructured surface. The crosslinker reacts with a portion of the azlactone groups of the copolymer, thereby forming the porous, crosslinked hydrogel. Unreacted azlactone groups in the hydrogel coating are then available for the attachment of functional materials for the appropriate end uses.

In addition to azlactone linking agents, copolymers including other linking agents can also be utilized. These include, for example, epoxy, carboxylic acid, hydroxyl, amine, N-hydroxysuccinimide, iso- and isothiocyanate, anhydride, aldehyde, and other groups, which are well known in the art for the immobilization of reactants. The copolymers comprising linking agents can be prepared by either step growth or chain growth polymerization processes as are well known in the art.

Azlactone moieties are useful because these moieties are suitable for reaction with numerous reactants, including oligonucleotides. Azlactone moieties are generally hydrolytically stable and therefore have a relatively long shelf life when used in applications of the present invention. These moieties also generally exhibit high reactivity with a wide variety of reactants.

The attachment chemistry layer can also be an ionic coating as disclosed in U.S. Pat. No. 6,783,838 (Coleman et al.), which is incorporated herein by reference. The ionic coating can include, for example, one or more ionic polymers, a hydrogel including hydrolyzed azlactone moieties, bifunctional molecules affixed to a hydrogel, or a hydrogel with an overcoating of one or more ionic polymers.

The ionic polymers can be either cationic or anionic. Suitable materials for providing a cationic polymeric coating include, but are not limited to, polymers and copolymers made from amine-containing monomers such as 2-vinylpyridine, 3-vinylpyridene, 4-vinylpyridene, (3-acrylamidopropyl)trimethylammonium chloride, 2-diethylaminoethyl acrylate, 2-diethylaminoethyl methacrylate, 3-dimethylaminopropyl acrylate, 3-dimethylaminopropyl methacrylate, 2-aminoethyl methacrylate, dimethylaminoethyl acrylate and methacrylate, 2-acryloxyethyltrimethylammonium chloride, diallyldimethylammonium chloride, 2-methacryloxyethyltrimethylammonium chloride, 3-methacryloxy-2-hydroxypropyltrimethylammonium chloride, 3-aminopropylmethacrylamide, dimethylaminoethyl methacrylamide, dimethylaminopropyl acrylamide, and other similarly substituted acrylamides and methacrylamides; 4-vinylbenzyltrimethylammonium chloride, 4-vinyl-1-methylpyridinium bromide, ethylene imine, lysine, allylamine, vinylamine, nylons and chitosan. Suitable materials for providing an anionic polymeric coating include but are not limited to polymers and copolymers of unsaturated acids such as acrylic, methacrylic, maleic, fumaric, itaconic, vinylbenzoic, N-acryloylamino, or N-methacryloylamino acids; 2-carboxyethyl acrylate; vinyl phosphoric acid; vinyl phosphonic acid; monoacryloxyethyl phosphate; sulfoethyl methacrylate; sulfopropyl methacrylate; 3-sulfopropyldimethyl-3-methacrylamidopropylammonium inner salt; styrenesulfonic acid; 2-acrylamido-2-methyl-1-propanesulfonic acid (AMPS); sulfonated polysaccharides such as heparin, dermatan sulfate, and dextran sulfate; carboxylated polyvinyl chloride; and carboxylated polysaccharides such as iduronic acid, carboxymethylcellulose or alginic acid.

In another embodiment, the ionic coating can include a hydrogel. As used herein, a hydrogel means a water-containing gel; that is, a polymer that is hydrophilic and will absorb water, yet is insoluble in water. A hydrogel can provide a porous surface coating capable of absorbing, for example, three to five times its dry weight in water. This provides a hydrophilic environment suitable for performing a wide variety of biological, chemical and biochemical assays.

In certain embodiments, the ionic coating can include linking agents. Useful linking agents include those described above. If desired, more than one type of linking agent can be used. When present, linking agents can be an integral component of the ionic coating, or can be affixed in a subsequent step to the ionic surface coating. Any number of processes known in the art can be used to introduce the linking agents to be affixed to the ionic coating. It is understood that the mode of affixation can vary in accordance with the linking agents employed.

In an alternative embodiment, the ionic coating comprises an ionic polymeric coating (the “ionic polymeric overcoating”) disposed on one of the ionic coatings described above (the “ionic surface coating”). The ionic polymeric overcoating can be cationic, anionic, or amphoteric. An ionic polymeric overcoating including an ionic surface may be desired to form ionic bonds with reactants so that the analytes subsequently can be detected or assayed.

The ionic polymeric overcoating can be used in conjunction with any surface coating. For example, an ionic polymeric overcoating can be applied to a nonionic surface coating, including a hydrogel comprising azlactone copolymers. In such an embodiment, it may be advantageous for the ionic polymeric coating to have functional groups that will covalently react with the azlactone polymer. Alternatively, the ionic polymeric overcoating can be applied to an ionic surface coating including a non-azlactone, ionic polymer. In such an embodiment, it may be advantageous to have a surface coating and an overcoating of opposite formal charge. In this way, the formal charges on the respective coatings will form ionic bonds between the surface coating and the overcoating. Additionally, there can be multiple overcoatings. The materials described above as being useful for a non-azlactone, ionic polymeric surface coating are equally suited for use in an ionic polymeric overcoating. Any of these materials can be crosslinked in the ionic polymeric overcoating. The ionic polymeric overcoating can be selected to provide the specific qualities desired for a particular application. For example, a cationic overcoating can be selected for an application in which the one or more anionic polypeptides (for example, proteins) are to be affixed.

In yet another embodiment, the ionic surface coating can include bifunctional small ionic molecules, such as amino-functional ionic molecules, affixed to linking agents. In the case of amino-functional ionic molecules, the amine forms a covalent bond with, for example, azlactone moieties in the linking agents of the surface coating. The ionic portion of the molecules provides the ionic surface coating with ionic character. The extent of the ionic character is determined by the particular ionic molecules selected for use. In this way, the ionic surface coating is provided with ionic character without requiring a polymeric overcoating. Any ionic molecule also having a functional group that is reactive with any portion of the linking agents can be suitable for the present invention. Suitable amine-functional ionic molecules include, but are not limited to, aminocarboxylic acids (for example, alpha.-, beta.-, gamma.-, etc. amino acids such as glycine, alanine, aspartic acid, .beta.-alanine, .gamma.-aminobutyric acid, and 12-aminododecanoic acid); aminosulfonic acids such as 2-aminoethane sulfonic acid (taurine) and 3-amino-1-propanesulfonic acid; aminophosphonic or phosphoric acids such as 2-aminoethanephosphonic acid, 2-aminoethyl dihydrogenphosphate, 2-aminoethyl thiophosphate sodium salt, and aminopropylphosphonic acid; and polyamines such as N,N-dimethylaminoethylamine, N,N-diethylaminopropylamine, N-aminopropylmorpholine, 2-(2-aminoethyl)pyridine, 2-aminoethyltrimethylammonium chloride, diethylenetriamine, triethylenetetraamine, tetraethylenepentaamine, 2-aminoethylpiperidine, and N-(2-aminoethyl) 1,3-propanediamine.

The attachment chemistry layer can also be a silicon-containing layer as disclosed in U.S. Pat. No. 6,881,538 (Haddad et al.), which is incorporated herein by reference. Silicon-containing layers for use in the present invention are preferably capable of silylation such that linking agents can be covalently bonded to the layer. It is believed that silylation can occur because of the presence of Si—OH groups, although this is not a necessary requirement. Such linking agents can be those traditionally used in functionalizing silica (for example, glass) surfaces. This material is suitable for the subsequent affixation of reactants thereto, although linking agents are not necessarily required for affixing reactants to a silicon-containing layer. The linking agents can be provided, for example, by functionalizing the silicon-containing layer with a coupling agent, or by coating a functionalized polymer thereon (for example, azlactone-functional polymers).

The type of functionalization will depend on the type of reactant(s). Preferably, a variety of conventional approaches to rendering the surfaces of silica materials chemically reactive are known and can be employed in the present invention to the extent their use creates linking agents on the silicon-containing layer for subsequent affixation of reactants. These include using silane coupling agents such as amino silanes to provide amino functionality, carboxy silanes to provide carboxy functionality, epoxy silanes to provide epoxy functionality, mercapto silanes (for example, those of the formula HS-L-Si(X)(Y)(Z) wherein L is divalent organic linking group, X is a hydrolyzable group such as alkoxy, acyloxy, amine or chlorine, Y and Z are hydrolyzable or nonhydrolyzable groups) to provide mercapto functionality, hydroxy silanes to provide hydroxy functionality, and the like. Conditions of such silylation reactions (that is, silanization reactions) are generally known to one of skill in the art. Examples of other silylation reactions are described in Van Der Voort et al., J. Liq. Chrom. & Rel Technol., 19, 2723-2752 (1996); Sudhakar Rao et al., Tet. Lett., 28, 4897-4900 (1987); Joos et al., Anal. Biochem., 247, 96-101 (1997); Aebersold et al., Anal Biochem., 187, 56-65 (1990); and PCT Publication WO 98/39481.

The silicon-containing layer can be a film or a coating. Films typically include plasma and/or vapor deposited materials containing silicon atoms such as, for example, silicon oxide films, silicon nitride films, silicon oxynitride film, plasma polymerized polysiloxane films, hydrogenated and nonhydrogenated amorphous silicon-containing films, silicon-doped diamond-like carbon films, and the like. See, for example, U.S. Pat. No. 6,696,157 (David et al.) and U.S. Pat. No. 6,795,636 (Cronk et al.), and Plasma Deposited Thin Films, J. Mort & F. Jansen, Eds.; CRC Press, Boca Raton, Fla. (1986). Coatings typically include materials containing silicon atoms deposited from a liquid, such as polysiloxanes, silicon oxides formed from hydrolysis reactions, and the like. Such silicon-containing layers provide a surface that can mimic silica (for example, glass) substrates with respect to reactivity and interaction with linking agents and reactants.

Preferred silicon-containing layers include diamond-like glass films. As the term is used herein, “diamond-like glass film” refers to substantially or completely amorphous films including carbon, silicon, and oxygen. The films can be covalently coupled or interpenetrating. The amorphous diamond-like films of this invention can contain clustering of atoms that give a short-range order but are essentially void of medium and long range ordering that lead to micro or macro crystallinity which can adversely scatter actinic radiation having wavelengths of from 180 nm to 800 nm. Diamond-like glass (DLG) includes an amorphous carbon system with a substantial quantity of silicon and oxygen, as in glass, yet still retains diamond-like properties. In these films, on a hydrogen-free basis, there is at least about 30% carbon, a substantial amount of silicon (at least about 25%) and not more than about 45% oxygen (references to compositional percentages herein refer to atomic percents). The unique combination of a fairly high amount of silicon with a significant amount of oxygen and a substantial amount of carbon makes these films highly transparent and flexible (unlike glass).

Microarrays of the invention comprising silicon-containing layers can also include polymeric coatings, typically overlying the silicon-containing layers, if desired. Such polymeric coatings can provide a variety of linking agents on the silicon-containing layer. Alternatively, they can be applied to a silicon-containing layer that already includes linking agents.

Suitable polymeric coatings include those that are suitable for affixing reactants and are compatible with the assays and attendant conditions that are to be conducted on the particular microarray. Examples include the polymeric coatings described in PCT Publication WO 99/53319. Suitable linking agents are azlactone moieties (such as those described above), epoxy, carboxylic acid, hydroxyl, amine, N-hydroxysuccinimide, iso- and isothiocyanate, anhydride, aldehyde, and other groups, which are well known in the art for the immobilization of reactants. Preferred polymeric coatings comprise copolymers prepared using hydrophilic or water-soluble comonomers such as acrylamide and acrylamide derivatives, hydroxyethylacrylate and methacrylate, and the like.

The attachment chemistry layer can also comprise N-sulfonylamidocarbonyl containing compounds or N-sulfonyldicarboximide containing compounds such as those described in U.S. Patent Application Publication Nos. 05/0107615 and 05/0227076, both of which are incorporated herein by reference.

For example, the attachment chemistry layer can comprise a tethering group attached to the microstructured surface, the tethering group comprising a reaction product of a complementary functional group G on the microstructured surface with a compound of formula I 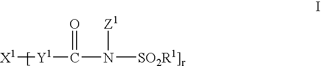
wherein
X¹ is a substrate-reactive functional group selected from a carboxy, halocarbonyl, halocarbonyloxy, cyano, hydroxy, mercapto, isocyanato, halosilyl, alkoxysilyl, acyloxysilyl, azido, aziridinyl, haloalkyl, tertiary amino, primary aromatic amino, secondary aromatic amino, disulfide, alkyl disulfide, benzotriazolyl, phosphono, phosphoroamido, phosphato, or ethylenically unsaturated group;
Y¹ is a single bond or a divalent group selected from an alkylene, heteroalkylene, arylene, carbonyl, carbonyloxy, carbonylimino, oxy, thio, —NR^{d}— where R^{d} is hydrogen or alkyl, or combinations thereof,
Z¹ is an alkyl, aryl, or —(CO)R^{a} wherein R^{a} together with R¹ and groups to which they are attached form a four to eight membered heterocyclic or heterobicyclic group having a nitrogen heteroatom and a sulfur heteroatom, wherein the heterocyclic or heterobicyclic group can be fused to an optional aromatic group, optional saturated or unsaturated cyclic group, or optional saturated or unsaturated bicyclic group;
R¹ is an alkyl, fluoroalkyl, chloroalkyl, aryl, NR^{b}R^{c} wherein R^{b} and R^{c} are each an alkyl group or taken together with the nitrogen atom to which they are attached form a four to eight membered cyclic group, or R¹ together with R^{a} and the groups to which they are attached form the four to eight membered heterocyclic or heterobicyclic group that can be fused to the optional aromatic group, optional saturated or unsaturated cyclic group, or optional saturated or unsaturated bicyclic group;
r is equal to 1 when X¹ is a monovalent group or equal to 2 when X¹ is a divalent group;
G is the complementary functional group capable of reacting with X¹ to form an ionic bond, covalent bond, or combinations thereof; and
the tethering group is unsubstituted or substituted with a halo, alkyl, alkoxy, or combinations thereof.

In another embodiment, the attachment chemistry layer comprises a tethering group attached to the microstructured surface, the tethering group comprising a reaction product of a complementary functional group G on the microstructured surface with a compound of formula II 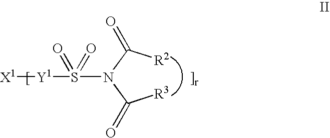
wherein
X¹ is a substrate-reactive functional group selected from a carboxy, halocarbonyl, halocarbonyloxy, cyano, hydroxy, mercapto, isocyanato, halosilyl, alkoxysilyl, acyloxysilyl, azido, aziridinyl, haloalkyl, tertiary amino, primary aromatic amino, secondary aromatic amino, disulfide, alkyl disulfide, benzotriazolyl, phosphono, phosphoroamido, phosphato, or ethylenically unsaturated group;
Y² is a single bond or a divalent group selected from an alkylene, heteroalkylene, arylene, carbonyl, carbonyloxy, carbonylimino, oxy, thio, or —NR^{a}—, or combinations thereof, wherein R^{a} is hydrogen, alkyl, or aryl;
R² and R³ together with a dicarboximide group to which they are attached form a four to eight membered heterocyclic or heterobicyclic group that can be fused to an optional aromatic group, optional saturated or unsaturated cyclic group, or optional saturated or unsaturated bicyclic group;
r is 1 when X¹ is a monovalent group or equal to 2 when X¹ is a divalent group;
G is the complementary functional group capable of reacting with X¹; and
the tethering group is unsubstituted or substituted with a halo, alkyl, alkoxy, or combinations thereof.

In yet another embodiment, the attachment chemistry layer comprises a tethering group attached to the microstructured surface, the tethering group comprising a reaction product of a complementary functional group G on the microstructured surface with a compound of formula III 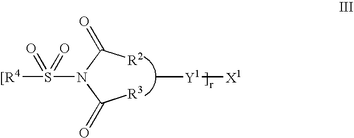
wherein
X¹ is a substrate-reactive functional group selected from a carboxy, halocarbonyl, halocarbonyloxy, cyano, hydroxy, mercapto, isocyanato, halosilyl, alkoxysilyl, acyloxysilyl, azido, aziridinyl, haloalkyl, tertiary amino, primary aromatic amino, secondary aromatic amino, disulfide, alkyl disulfide, benzotriazolyl, phosphono, phosphoroamido, phosphato, or ethylenically unsaturated group;
R² and R³ together with a dicarboximide group to which they are attached form a four to eight membered heterocyclic or heterobicyclic group that can be fused to an optional aromatic group, optional saturated or unsaturated cyclic group, or optional saturated or unsaturated bicyclic group;
Y¹ is a single bond or a divalent group selected from alkylene, heteroalkylene, arylene, carbonyl, carbonyloxy, carbonylimino, oxy, thio, —NR^{d}— where R^{d} is hydrogen or alkyl, or combinations thereof,
R⁴ is an alkyl, aryl, aralkyl, or —NR^{b}R^{c} wherein R^{b} and R^{c} are each an alkyl group or taken together with the nitrogen atom to which they are attached form a four to eight membered heterocyclic group;
r is equal to 1 when X¹ is monovalent or equal to 2 when X¹ is a divalent group;
G is the complementary functional group capable of reacting with X²; and
the tethering group is unsubstituted or substituted with a halo, alkyl, alkoxy, or combinations thereof.

In general, the type of functionalization of the attachment chemistry layer will depend on the type of microstructured surface utilized and the reactant(s). One skilled in the art will appreciate that a variety of approaches to rendering the surfaces of polymeric materials chemically reactive are known and can be employed in the present invention to the extent their use creates linking agents for subsequent affixation of reactants.

The attachment chemistry layer can be applied to the microstructured surface by any suitable means known in the art. Appropriate application means will, of course, depend on the type of attachment chemistry employed.

Some attachment chemistry layers such as, for example, azlactone layers, ionic coatings, N-sulfonyldicarboximide containing compounds, and N-sulfonylaminocarbonyl containing compounds can be applied by conventional means known in the art such as, for example, extrusion coating, die coating, dip coating, air-knife coating, gravure coating, curtain coating, spray coating, use of wire wound coating rods, and the like.

In some embodiments, the attachment chemistry layer can be crosslinked or otherwise treated, for example, to insolubilize, modify the glass transition temperature (Tg), or modify the adhesion properties of the coating. For example, copolymers that have a low Tg can be formulated with a cross-linker in order to raise the Tg of the resultant coating.

Typically, the coating is between about 0.01 and about 10 microns. Coatings less than about 1 micron are preferred in order to minimize diffusion difficulties that may arise when using thicker coatings. An analyte of interest may have to diffuse through the attachment chemistry layer prior to contacting a reactant affixed thereto. If the coating is relatively thick, for example greater than about 10 microns, the diffusion time required could slow the kinetics of the analyte/reactant interaction.

Adhesion of the coating to the substrate (that is the microstructured surface or any intervening layer) can be improved, if desired, by any of the methods known to one skilled in the art. These methods include various pre-treatments to or coatings on the microstructured surface such as, for example, corona or plasma treatment, or by application of primers. Suitable primers can include, without limitation, polyethylenimine, polyvinylidenechloride, primers such as those described in U.S. Pat. No. 5,602,202 (Groves), the disclosure of which is incorporated herein by reference, and colloidal dispersions of inorganic metal oxides in combination with ambifunctional silanes such as described in U.S. Pat. No. 5,204,219 (Van Ooij et al.), U.S. Pat. No. 5,464,900 (Stotko, Jr. et al.), and U.S. Pat. No. 5,639,546 (Bilkadi), the disclosures of which are all incorporated herein by reference. Other methods of increasing adhesion of copolymers to polyolefin microstructured surfaces are disclosed in U.S. Pat. No. 5,500,251 (Burgoyne, Jr. et al.), the disclosure of which is incorporated herein by reference.

Other attachment chemistry layers such as, for example, diamond-like films, can be deposited by plasma deposition from gases. Methods and apparatus for depositing diamond-like films are disclosed in U.S. Pat. No. 6,696,157 (David et al.) and U.S. Pat. No. 6,795,636 (Cronk et al.).

Reactants can be affixed to the attachment chemistry layer to create binding sites. Any number of processes known in the art can be used to introduce the reactants to be affixed to the attachment chemistry layer. It is understood that the mode of affixation can vary in accordance with the reactant or reactants employed.

The type of reactant used in the present invention will vary according to the application and the analyte of interest. For example, when characterizing DNA, oligonucleotides are preferred. When conducting diagnostic tests to determine the presence of an antigen, antibodies are preferred. Accordingly, suitable reactants include, without limitation, amino acids, nucleic acids, including oligonucleotides and cDNA, carbohydrates, and proteins such as enzymes and antibodies.

Reactants can be introduced to the attachment chemistry layer of the microarrays of the invention for affixation to create binding sites. The modes of affixation can include, without limitation, covalent and ionic bonding, adherence, and physical entrapment of the reactants. Regardless of how the reactants affix, any number of processes known in the art can be used to introduce the reactants to the attachment chemistry layer, including on-chip or off-chip synthesis. Solutions containing reactants to be affixed can be simultaneously introduced by arrays of capillary tubes, by arrayed pipetting devices, or by an array of posts designed to transfer liquid droplets from a tray of reservoirs.

Analytes can be detected in a sample of interest by contacting the sample with a reactant affixed to the attachment chemistry layer, and detecting any complexes formed from the binding of the analyte to the reactant. The presence or amount of complexes can be related to the presence or amount of analyte in the sample.

Analytes can also be detected in a sample by contacting the sample with a reactant affixed to the attachment chemistry layer to form a complex, and then contacting the complex with a second reactant (for example, an enzyme-linked antibody capable of generating multiple reporter molecules) to form a ternary complex. The ternary complexes can be detected and the presence or amount of ternary complexes can be related to the presence or amount of analyte in the sample. The complexes can be detected, for example, by fluorescence, absorbance, electric current, chemiluminescence, or the like. Generally, this is accomplished by addition of a solution containing a substrate for the enzyme. Action of the enzyme on the substrate generates a reporter molecule that can be measured by any of the techniques listed above.

Preferably, the sample is allowed to incubate with the substrate solution for a time sufficient to obtain multiple turnovers of the substrate, thus increasing the amount of reporter molecule generated within each microstructured feature. The microstructured elements of the microarrays of the invention confine the sample during the incubation period, and prevent the sample from diffusing beyond the microstructured element. The microarrays of the invention therefore provide a higher sensitivity than can be achieved using planar microarrays in which the reporter molecule diffuses away from the feature where it was deposited.

Optional Layers and Features

The microarrays of the invention can be provided on a substrate. The substrate can support the microarray during manufacturing and/or use. Useful substrate materials include organic and inorganic materials. For example, the substrate can comprise inorganic glasses, ceramic materials, polymeric materials, filled polymeric materials, or fibrous materials. The microarray can be attached to the substrate using any suitable means such as, for example, using an adhesive.

In some embodiments, the microarrays of the invention can further comprise reference or “fiducial” markings located at one or more predetermined positions on or inside the microarray. Fiducial markings can be sensed by an automated system in order to provide the capability of real-time monitoring of the position of the microarray during manufacturing. Fiducial markings can also aid in identifying the locations of the microstructured elements during image processing. A fiducial mark can be, for example, a laser-etched region, an imprinted geometric shape or lettering, an optical signal, or a fluorescent marker.

In certain embodiments, the microarrays of the invention can include an optional layer. The optional layer can include, for example, a mask layer to reduce or prevent transmission of excitation energy through the mask layer to an underlying layer or substrate, as reported in PCT Publication WO 01/16370. For other applications, a mask layer can be used to reduce or prevent the transmission of electromagnetic energy from beneath the analyte that is similar to the electromagnetic signal emitted by the desired analyte in response to excitation energy. In either case, with a mask layer in place, the electromagnetic signals emitted from the surface of the microarray can generally be attributed to excitation of the molecule captured on the microarray rather than an underlying layer or substrate.

The optional layer can alternatively include an electromagnetic energy sensitive material, which may be the same or different than the material of the mask layer, if present. The optional layer including electromagnetic energy sensitive material can take a variety of forms as reported in U.S. Pat. No. 6,482,638 (Patil et al.). Examples of some suitable materials include, but are not limited to, those reported in U.S. Pat. No. 5,278,377 (Tsai), U.S. Pat. No. 5,446,270 (Chamberlain et al.), U.S. Pat. No. 5,529,708 (Palmgren et al.), and U.S. Pat. No. 5,925,455 (Bruzzone et al.). The optional layer can be in direct contact with the microstructured surface, or one or more intervening layers can be located between the optional layer and microstructured surface.

For some applications, it is advantageous to use a cover with the microarrays of the invention. A cover can, for example, protect samples from the environment (for example, dust) and/or keep the samples from evaporating.

### EXAMPLES

Objects and advantages of this invention are further illustrated by the following examples, but the particular materials and amounts thereof recited in these examples, as well as other conditions and details, should not be construed to unduly limit this invention.

Unless otherwise noted, all reagents were or can be obtained from Sigma-Aldrich Corp., St. Louis, Mo.

As used herein,

“CHES buffer” refers to an aqueous solution of 2-(cyclohexylamino)ethanesulfonic acid;

“SA-HRP” refers to streptavidin conjugated with horseradish peroxidase, which was obtained from Jackson ImmunoResearch Laboratories, Inc., West Grove, Pa.;

“ABTS” refers to 2,2′-azino-di-(3-ethylbenzthiazoline-6-sulfonate), which was obtained in kit form from KPL Inc., Gaithersburg, Md.;

“TWEEN 20” refers to polyoxyethylene(20)sorbitan monolaurate; and

“SDS” refers to sodium dodecyl sulfate.

### Preparative Example 1

Preparation of a Film Having a Microstructured Surface

A film having a microstructured surface was prepared by extruding DOW 7C50 resin (DOW 7C50, manufactured by The Dow Chemical Co., Midland, Mich.) and drawing the molten extruded resin between two approximately 30.5 centimeter (12 inch) diameter cylindrical nip rolls. The upper nip roll was a rubber-coated roll and the lower nip roll was a metal roll with a repeating pattern engraved on its surface.

This repeating pattern included two sets each of primary and secondary grooves where each set contained a pair of grooves that were parallel to each other. Each member of grooves within the primary set was spaced approximately 250 micrometers (0.0098 inch) from the other member of the pair. The two sets of primary grooves were perpendicular to each other. The first and second set of primary grooves had depths of approximately 32 micrometers (0.00126 inch) and 36 micrometers (0.00142 inch), respectively, and had widths of approximately 18 micrometers (0.00071 inch) at the bottom of each groove and approximately 27 micrometers (0.00106 inch) at the top of each groove. Each member within a set of secondary grooves was spaced approximately 25 micrometers (0.00098 inch) from the other member of the pair. The first and second set of secondary grooves were perpendicular to each other. The first and second sets of secondary grooves had depths of approximately 3 micrometers (0.00012 inch) and 5 micrometers (0.00019 inch), respectively, and had widths of approximately 5 micrometers (0.000019 inch) at the bottom of each groove and approximately 7 micrometers (0.00027 inch) at the top of each groove.

The DOW 7C50 resin was extruded using a Killion single screw extruder (available from Davis-Standard Killion, Pawcatuck, Conn.) having a diameter of 3.18 centimeters (1.25 inches), a length to diameter ratio of 30 to 1, and five heated zones, the temperatures of which were set to 124° C., 177° C., 235° C., 243° C., and 249° C., respectively. The extrusion temperature was set to 249° C. The die was placed in proximity to the nip rolls so that the molten extruded resin was drawn between the nip rolls as it exited the die. The temperature of the upper rubber-coated roll was set to 10° C. and the temperature of the lower engraved metal roll was set to 66° C. The web speed was approximately 2.7 meters (8.8 feet) per minute. The pattern of the engraved metal nip roll was transferred to the extruded polypropylene film so that the surface of the film was microstructured with the inverse of the pattern that was engraved on the roll. The film had primary microstructures that corresponded to the primary grooves on the engraved metal nip roll, and had secondary microstructures that corresponded to the secondary grooves on the engraved metal nip roll. The primary microstructures were oriented approximately 45° with respect to the long axis of the film (that is, approximately 45° to the web direction). The secondary microstructures were oriented approximately 45° to the primary microstructures. The film had a thickness of approximately 142 micrometers (0.0056 inch). A scanning electron micrograph of a section of the surface of the film is shown in FIG. 3.

A low fluorescence transfer adhesive (3M Company, St. Paul, Minn.) was laminated to the non-structured side of the microstructured polypropylene film. Then, with the aid of a stereomicroscope to align the edges of a cutting die with the primary microstructures on the surface of the film, it was then cut to 2 centimeter (0.79 inch) by 6 centimeter (2.36 inches) pieces. The adhesive side of the film was then centered on a 2.54 centimeter (1 inch) by 7.62 centimeter (3 inches) glass microscope slide and was pressed onto the slide using a small hydraulic press. The glass slide was then placed in a robotic microarrayer (Cartesian Dispensing Systems, available from Genomic Solutions, Ann Arbor, Mich.).

A one weight percent aqueous solution of a monoreactive fluorescent dye (CY5, obtained from Amersham Biosciences Corp., Piscataway, N.J.) was deposited on the microstructured surface of the substrate using contact spotting with a microarray printing pin (200 micrometer tip diameter, available from Genetix USA, Inc., Boston, Mass.) to produce three six by six patterns in every third primary microstructure. The deposited solution was allowed to dry at room temperature, and then the slide was placed in a Model LS300 microarray scanner (obtained from Tecan US, Durham, N.C.) and was imaged according to the directions supplied by the manufacturer. The resulting pattern is shown in FIG. 4.

### Preparative Example 2

Preparation of an Azlactone-Containing Polymer

A reaction vessel, fitted with a mechanical stirrer, thermometer, and reflux condenser, was charged with 12 parts by weight 2-vinyl-4,4-dimethyl-2-oxazolin-5-one (vinyldimethylazlactone, available from TCI America, Portland, Oreg.), 28 parts by weight N,N-dimethylacrylamide, 0.15 parts by weight 2,2′-azobisisobutyronitrile (AIBN), and 60 parts by weight toluene. Nitrogen gas was bubbled through the mixture and then the mixture was heated to 55° C. and was stirred under a nitrogen atmosphere for 24 hours. The mixture was then cooled to room temperature and diluted with an additional 60 parts by weight of 2-propanol. Gravimetric analysis showed that the product had a solids content of 24.2 weight percent.

### Example 1

Microarray Having a Microstructured Surface and an Attachment Chemistry Layer

The azlactone-containing polymer solution of Preparative Example 2 was diluted with 2-propanol to provide 10 grams of a mixture that had a solids content of 0.75 weight percent. This mixture was coated onto the microstructured polypropylene film of Preparative Example 1 using a #14 wire-wound coating rod to provide a wet thickness of about 0.03 millimeters. Ethylenediamine (10.6 microliters), sufficient to react with a third of the azlactone groups, was mixed with the polymer solution just before the mixture was coated on the film. The coated film was dried in a forced air oven at 55° C. for approximately 30 minutes. The surface of the film including the attachment chemistry layer was analyzed by attenuated total reflection infrared spectroscopy (ATR-IR) and the presence of the azlactone carbonyl group (weak absorption at about 1820 cm⁻¹) and the amide carbonyl group (absorption at about 1650 cm⁻¹) was observed.

### Example 2

Enzyme-Linked Assay Using a Microreplicated Microarray Having an Attachment Chemistry Layer

A buffered solution of an immunoglobin (20 micrograms per milliliter of anti-human mouse IgG in CHES buffer) is deposited on the microstructured surface of a film prepared as described in Preparative Example 1 using contact spotting with a microarray printing pin (200 micrometer tip diameter, available from Genetix USA, Inc., Boston, Mass.) to produce three six by six patterns in every primary microstructure (hereinafter, the “loaded microstructures”). This microstructured film is then allowed to stand for 60 minutes and is then rinsed three times with PBS buffer that contains 0.05 weight percent TWEEN 20. The microstructured film is then allowed to dry at room temperature. Each of the loaded microstructures is then filled with a 2 weight percent nonfat dry milk powder (available under the trade designation “NESTLE CARNATION NONFAT DRY MILK POWDER” from Nestle USA, Glendale, Calif.) in PBS buffer. This microstructured film is then allowed to stand for 60 minutes and is then rinsed three times with PBS buffer that contained 0.05 weight percent TWEEN 20. Into each of the loaded microstructures is then deposited a solution of 4 micrograms per milliliter of biotin-conjugated human IgG in PBS buffer. This microstructured film is then allowed to stand for 60 minutes and is then rinsed three times with PBS buffer that contained 0.05 weight percent TWEEN 20. Then into each of the loaded microstructures is deposited a solution of 0.5 micrograms per milliliter of the detecting enzyme SA-HRP in PBS buffer. This microstructured film is then allowed to stand for 30 minutes and is then rinsed three times with PBS buffer that contained 0.05 weight percent TWEEN 20. The ABTS indicator solution is then deposited into each loaded microstructure and, after approximately 5 minutes, a one weight percent aqueous solution of SDS is deposited into each loaded microstructure. This microarray is then analyzed.

Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

We claim:

1. A microarray comprising a microstructured surface and an attachment chemistry layer disposed on at least a portion of the microstructured surface, the microstructured surface comprising primary microstructured elements comprising walls.

2. The microarray of claim 1 wherein the attachment chemistry layer is a coating on the microstructured surface.

3. The microarray of claim 1 wherein the attachment chemistry layer is a functionalized portion of the microstructured surface.

4. The microarray of claim 1 wherein the microstructured surface comprises polyolefin.

5. The microarray of claim 1 wherein the walls have a thickness of between about 1 and about 50 micrometers.

6. The microarray of claim 1 wherein the walls have a height of between about 5 and about 200 micrometers.

7. The microarray of claim 1 wherein the pitch of the primary microstructured elements is between about 1 and about 1,000 micrometers.

8. The microarray of claim 7 wherein the pitch of the primary microstructured elements is between about 20 and about 200 micrometers.

9. The microarray of claim 1 wherein the primary microstructured elements have a volume of between about 1 to about 20,000 pL.

10. The microarray of claim 1 wherein the primary microstructured elements are cube elements.

11. The microarray of claim 1 wherein a base surface extends between the walls of the primary microstructured elements, and the base comprises secondary microstructured elements having an x-direction dimension.

12. The microarray of claim 11 wherein the x-direction dimension of the secondary microstructured elements is at least about 5 micrometers less than the height of the walls of the primary microstructured elements.

13. The microarray of claim 12 wherein the x-direction dimension of the secondary microstructured elements is at least about 50 micrometers less than the height of the walls of the primary microstructured elements.

14. The microarray of claim 11 wherein the secondary microstructured elements extend from one wall to a second wall.

15. The microarray of claim 1 wherein the attachment chemistry layer comprises linking agents.

16. The microarray of claim 15 wherein the linking agents comprise an azlactone moiety.

17. The microarray of claim 1 wherein the attachment chemistry layer comprises a crosslinked hydrogel comprising at least one azlactone-functional copolymer.

18. The microarray of claim 1 wherein the attachment chemistry layer has an ionic surface.

19. The microarray of claim 18 wherein the attachment chemistry layer comprises one or more ionic polymers, a hydrogel including hydrolyzed azlactone moieties, bifunctional molecules attached to a hydrogel, or a hydrogel with an overcoating of one or more ionic polymers.

20. The microarray of claim 1 wherein the attachment chemistry layer is a silicon-containing layer.

21. The microarray of claim 20 wherein the attachment chemistry layer is capable of silylation such that linking agents can be covalently bonded to the attachment chemistry layer.

22. The microarray of claim 20 wherein the silicon-containing layer is functionalized with a coupling agent or with a functionalized polymer coating.

23. The microarray of claim 20 wherein the attachment chemistry layer is a diamond-like glass film.

24. The microarray of claim 1 wherein the attachment chemistry layer comprises a tethering group attached to the microstructured surface, the tethering group comprising a reaction product of a complementary functional group G on the microstructured surface with a compound of formula I 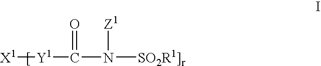
wherein
X¹ is a substrate-reactive functional group selected from a carboxy, halocarbonyl, halocarbonyloxy, cyano, hydroxy, mercapto, isocyanato, halosilyl, alkoxysilyl, acyloxysilyl, azido, aziridinyl, haloalkyl, tertiary amino, primary aromatic amino, secondary aromatic amino, disulfide, alkyl disulfide, benzotriazolyl, phosphono, phosphoroamido, phosphato, or ethylenically unsaturated group;
Y¹ is a single bond or a divalent group selected from an alkylene, heteroalkylene, arylene, carbonyl, carbonyloxy, carbonylimino, oxy, thio, —NR^{d}— where R^{d} is hydrogen or alkyl, or combinations thereof;
Z¹ is an alkyl, aryl, or —(CO)R^{a} wherein R¹ together with R¹ and groups to which they are attached form a four to eight membered heterocyclic or heterobicyclic group having a nitrogen heteroatom and a sulfur heteroatom, wherein the heterocyclic or heterobicyclic group can be fused to an optional aromatic group, optional saturated or unsaturated cyclic group, or optional saturated or unsaturated bicyclic group;
R¹ is an alkyl, fluoroalkyl, chloroalkyl, aryl, NR^{b}R^{c} wherein R^{b} and R^{c} are each an alkyl group or taken together with the nitrogen atom to which they are attached form a four to eight membered cyclic group, or R¹ together with R^{a} and the groups to which they are attached form the four to eight membered heterocyclic or heterobicyclic group that can be fused to the optional aromatic group, optional saturated or unsaturated cyclic group, or optional saturated or unsaturated bicyclic group;
r is equal to 1 when X¹ is a monovalent group or equal to 2 when X¹ is a divalent group;
G is the complementary functional group capable of reacting with X¹ to form an ionic bond, covalent bond, or combinations thereof; and
the tethering group is unsubstituted or substituted with a halo, alkyl, alkoxy, or combinations thereof.

25. The microarray of claim 1 wherein the attachment chemistry layer comprises a tethering group attached to the microstructured surface, the tethering group comprising a reaction product of a complementary functional group G on the microstructured surface with a compound of formula II 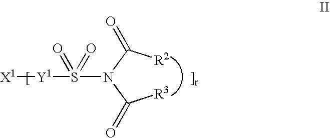
wherein
X¹ is a substrate-reactive functional group selected from a carboxy, halocarbonyl, halocarbonyloxy, cyano, hydroxy, mercapto, isocyanato, halosilyl, alkoxysilyl, acyloxysilyl, azido, aziridinyl, haloalkyl, tertiary amino, primary aromatic amino, secondary aromatic amino, disulfide, alkyl disulfide, benzotriazolyl, phosphono, phosphoroamido, phosphato, or ethylenically unsaturated group;
Y² is a single bond or a divalent group selected from an alkylene, heteroalkylene, arylene, carbonyl, carbonyloxy, carbonylimino, oxy, thio, or —NR^{a}—, or combinations thereof, wherein R^{a} is hydrogen, alkyl, or aryl;
R² and R³ together with a dicarboximide group to which they are attached form a four to eight membered heterocyclic or heterobicyclic group that can be fused to an optional aromatic group, optional saturated or unsaturated cyclic group, or optional saturated or unsaturated bicyclic group;
r is 1 when X¹ is a monovalent group or equal to 2 when X¹ is a divalent group;
G is the complementary functional group capable of reacting with X¹; and
the tethering group is unsubstituted or substituted with a halo, alkyl, alkoxy, or combinations thereof.

26. The microarray of claim 1 wherein the attachment chemistry layer comprises a tethering group attached to the microstructured surface, the tethering group comprising a reaction product of a complementary functional group G on the microstructured surface with a compound of formula III 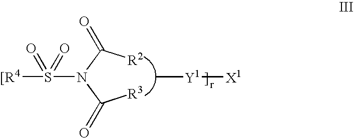
wherein
X¹ is a substrate-reactive functional group selected from a carboxy, halocarbonyl, halocarbonyloxy, cyano, hydroxy, mercapto, isocyanato, halosilyl, alkoxysilyl, acyloxysilyl, azido, aziridinyl, haloalkyl, tertiary amino, primary aromatic amino, secondary aromatic amino, disulfide, alkyl disulfide, benzotriazolyl, phosphono, phosphoroamido, phosphato, or ethylenically unsaturated group;
R² and R³ together with a dicarboximide group to which they are attached form a four to eight membered heterocyclic or heterobicyclic group that can be fused to an optional aromatic group, optional saturated or unsaturated cyclic group, or optional saturated or unsaturated bicyclic group;
Y¹ is a single bond or a divalent group selected from alkylene, heteroalkylene, arylene, carbonyl, carbonyloxy, carbonylimino, oxy, thio, —NR^{d}— where R^{d} is hydrogen or alkyl, or combinations thereof,
R⁴ is an alkyl, aryl, aralkyl, or —NR^{b}R^{c} wherein R^{b} and R^{c} are each an alkyl group or taken together with the nitrogen atom to which they are attached form a four to eight membered heterocyclic group;
r is equal to 1 when X¹ is monovalent or equal to 2 when X¹ is a divalent group;
G is the complementary functional group capable of reacting with X²; and
the tethering group is unsubstituted or substituted with a halo, alkyl, alkoxy, or combinations thereof.

27. The microarray of claim 1 wherein the attachment chemistry layer is suitable for subsequent affixation of a reactant selected from the group consisting of amino acids, nucleic acids, carbohydrates, and proteins.

28. The microarray of claim 27 wherein the attachment chemistry layer is suitable for subsequent affixation of a reactant selected from the group consisting of DNA, enzymes, and antibodies.

29. The microarray of claim 1 wherein a reactant is affixed to the attachment chemistry layer.

30. The microarray of claim 1 further comprising fiducial markings that can be sensed by an automated system.

31. A kit comprising the microarray of claim 1 and a cover.

32. A method for detecting analytes in a sample comprising:
(a) providing a microarray according to claim 29;
(b) depositing a sample into at least one primary microstructured element of the microarray such that the sample contacts the reactant and forms a complex;
(c) detecting any complexes; and
(d) relating the presence or amount of the complexes to the presence or amount of analyte in the sample.

33. A method for detecting analytes in a sample comprising:
(a) providing a microarray according to claim 29;
(b) depositing a sample into at least one primary microstructured element of the microarray such that the sample contacts the reactant and forms a complex;
(c) contacting the complex with a second reactant to form a ternary complex;
(d) detecting any ternary complexes; and
(e) relating the presence or amount of the ternary complexes to the presence or amount of analyte in the sample.
